(19) United States
(12) Patent Application Publication
(10) Pub. No.: US 2026/0049344 A1
(43) Pub. Date: Feb. 19, 2026

(54) ENUMERATING MICROORGANISMS USING GELABLE, COLD WATER-SOLUBLE MEDIA

(71) Applicant: Institute for Environmental Health, Inc., Lake Forest Park, WA (US)

(72) Inventor: Mansour Samadpour, Lake Forest Park, WA (US)

(21) Appl. No.: 19/101,548

(22) PCT Filed: Aug. 23, 2023
(86) PCT No.: PCT/US2023/072757

§ 371 Date: Feb. 5, 2025

Related U.S. Application Data
(60) Provisional application No. 63/402,896, filed on Aug. 31, 2022.

(51) Int. Cl. C12Q 1/06; C12N 1/20; C12R 1/01

(52) U.S. Cl. CPC C12Q 1/06; C12N 1/20; C12R 2001/01

(57) ABSTRACT

Provided are methods for enumerating target microorganisms in a test sample, comprising: retaining a liquid test sample having dissolved therein a nutrient base media component and a glucomannan-containing gelling agent component of a solid cold water-soluble microbiological medium; forming a gelled layer of the dissolved medium; incubating the gelled layer under conditions sufficient for microbial growth and detectable colony formation in and/or on the gelled layer; and enumerating a number of target microorganisms in the test sample by counting the colonies. Preferred gelling agents comprises 5-7 g glucomannan per 100 ml liquid test sample, and optionally comprise a gum(s). Compositions, kits and combinations comprising a nutrient base media and a glucomannan-containing gelling agent, are also provided. The compositions and methods enable enumerating a small number of target microorganisms (e.g., <1 cell/mL) in a large volume (e.g., 50 ml to 150 mL or larger) of a liquid test sample.

### CROSS REFERENCE TO THE RELATED APPLICATIONS

This application claims priority to U.S. Patent Application No. 63/402,896, filed Aug. 31, 2022, entitled “ENUMERATING MICROORGANISMS USING GELABLE, COLD WATER-SOLUBLE MEDIA”, which is hereby incorporated herein by reference in their entirety and for all purposes.

### FIELD OF THE INVENTION

Aspects of the invention relate generally to compositions and methods for enumerating microorganisms present in a test sample, more particularly to the use of microbiological media comprising a cold water-soluble gelling agent, nutrients (e.g., a nutrient base media), and an indicator reagent (e.g., a color-developing agents, chromogenic substrates, etc.) to enumerate a level of target organisms in a volume of test sample, and most particularly to enumerate a low level (e.g., less than 1 cell/mL) of target organisms in a high volume (e.g., 5 ml, 10 ml, 50 ml, 100 ml, 150 ml, 500 ml, 1000 ml, etc.) of test sample.

### BACKGROUND

Conventionally, bacterial enumeration is performed using pour plate methods, surface spread plate methods and spiral plate methods; all of which typically utilize a relatively small volume (e.g., 0.1 mL to 1 mL) of a test sample. For certain samples (e.g., water samples, milk samples, beverage, food, etc.), however, when a low number of a target organism (e.g., less than 1 cell/mL) is expected in a test sample, a relatively large volume of the test sample (e.g., for water samples typically 100 mL) is utilized, and in such cases, a most probable number (MPN) procedure (e.g., BAM Appendix 2: Most Probable Number from Serial Dilutions, 2020) or a membrane filter technique is used for the enumeration. The MPN procedure is not a precise measure since it is determined by comparing the pattern of positive results with a statistical table. Moreover, the MPN method is laborious and time consuming, and not readily adaptable for use in the field. The membrane filter technique can provide more rapid and precise quantitative data but is relatively expensive and time consuming since it requires a filtration apparatus and media preparation, and often is not applicable to turbid samples.

Likewise, some food samples, e.g., low water activity foods such as spices, are toxic to certain bacteria when they are wet, and in the absence of methods for neutralizing the toxicity, diluting such foods beyond their toxic levels to examine them for the presence of indicator organisms, surrogate organisms and pathogens is recommended-resulting in large sample sizes. For example, it is recommended that allspice, cinnamon, and oregano are diluted at 1:100 sample/broth ratio, and cloves at 1:1000 sample/broth ratio. Moreover, spices have been implicated in outbreaks of Salmonellosis, and processes for the inactivation of Salmonella require a 5-log reduction of Salmonella on the spices. Furthermore, to calculate the log reduction, the count of the Salmonella or Salmonella's surrogate per weight unit (e.g., gram) of spice needs to be determined, which equates to determining the counts of the target organism per 100 ml diluents for allspice, cinnamon, and oregano or 1000 ml diluents for cloves.

There is a need in the art, therefore, for a rapid and easy-to-use enumeration method which allows enumerating a low level of a target organisms in a high volume of test sample (e.g., as low as 1 cell of the target organism in e.g., 5 ml, 10 mL, 50 ml, 100 mL, 150 ml, 500 ml, or 1000 mL, etc., volume of test sample).

### SUMMARY OF EXEMPLARY ASPECTS OF THE INVENTION

Embodiments of the disclosure can be described in view of the following clauses:

1. A method for enumerating a target organism in a test sample, comprising: retaining, in a suitable container, a liquid test sample having dissolved therein a nutrient base media component and a glucomannan-containing gelling agent component of a gelable, solid cold water-soluble microbiological medium; forming, in the container, a gelled layer of the dissolved medium within a gelling time period of less than 1 hour after dissolution of the gelling agent component in the liquid test sample; incubating the gelled layer at one or more incubation temperature(s) and for a time sufficient to provide for microbial growth and detectable microbiological colony formation in or on the gelled medium layer; and counting the colonies to enumerate a number of target microorganisms in the test sample, wherein less than 1 target organism/mL of test sample may be detected and enumerated, with no requirement for filtering or concentrating the liquid test sample.
2. The method of clause 1, wherein the test sample is a volume in the range of from about 50 ml to about 150 ml.
3. The method of clause 1 or 2, wherein the gelling time period is within about 1 to about 10 minutes, within about 1 to 5 minutes, within about 2 to about 5 minutes, or within about 3 to about 5 minutes, in each case after combining the gelling agent component with the liquid test sample.
4. The method of any one of clauses 1-3, wherein the dissolution and forming are at one or more temperature(s) in a range of 10° C. (50° F.) to 45° C. (113° F.).
5. The method of any one of clauses 1-4, wherein the amount of glucomannan is from about 4% to about 10% (wt./wt.), from about 5% to about 10% (wt./wt.), or from about 5% to about 7% (wt./wt.) of the liquid test sample.
6. The method of any one of clauses 1-5, wherein, in addition to the glucomannan, the gelling agent component comprises at least one of a gum, a natural polymer, and/or synthetic polymer.
7. The method of clause 6, wherein the gum comprises guar gum and/or xanthan gum.
8. The method of any one of clauses 1-7, wherein, prior to dissolution in the liquid test sample, the glucomannan of the glucomannan-containing gelling agent component of the gelable, solid cold water-soluble microbiological medium has been heat cured at a temperature and for a time sufficient to remove or substantially remove water therefrom and/or provide adequate sterilization thereof.
9. The method of any one of clauses 1-8, wherein the dissolution and forming are at room temperature(s) or ambient temperature(s) in a range of about 16° C. (61° F.) and about 25° C. (77° F.).
10. The method of any one of clauses 1-9, wherein the incubation temperature is equal to, or greater than, the dissolution and forming temperature.
11. The method of any one of clauses 1-10, wherein the gelled layer has a thickness of about 1 to about 5 mm, about 2 to about 5 mm, or about 2 to about 3 mm, and/or wherein the container is transparent.
12. The method of any one of clauses 1-11, wherein the gelled layer comprises a marker agent, and/or an antibiotic, and/or an inhibiting agent.
13. The method of clause 12, wherein the marker is a colorimetric or a fluorescent marker.
14. The method of any one of clauses 1-13, wherein the nutrient base media component and the gelling agent component are separate components that are dissolved in the liquid test sample by dissolving them serially or concurrently, or wherein the nutrient base media component and the gelling agent component are in the form of a mixed component that is dissolved in the liquid test sample.
15. The method of any one of clauses 1-14, wherein the nutrient base media component and the gelling agent component are separate components, and the dissolving of the nutrient base media component in the liquid test sample precedes the dissolving of the gelling agent component in the liquid test sample.
16. The method of clause 15, wherein the nutrient base media is a component of the liquid test sample.
17. The method of any one of clauses 1-16, wherein an inner surface of the sample container is pre-coated with the solid cold water-soluble gelling agent(s) and/or the solid nutrient base medium, and retaining comprises initially introducing the liquid test sample into the pre-coated sample container.
18. The method of any one of clauses 1-17, wherein after forming of the gelled layer, the formed gel layer spontaneously softens within a softening period of from about 2 to about 10 days, from about 2 to about 5 days, from about 3 to about 5 days, from about 4 to about 5 days, or within about 5 days.
19. A gelled liquid test sample, comprising a gelled layer of a liquid test sample containing a dissolved nutrient base medium and a dissolved glucomannan-containing gelling agent, wherein the gelled layer has a thickness of about 1 to about 5 mm, wherein the amount of glucomannan is from about 4% to about 10% (wt./wt.) of the liquid test sample, and wherein the gelled layer of the liquid test sample is suitable to provide for microbial growth and detectable microbiological colony formation therein and/or thereon.
20. The gelled liquid test sample of clause 19, wherein the gelled layer of the liquid test sample has a volume in the range of from about 50 ml to about 150 ml.
21. The gelled liquid test sample of clause 19 or 20, wherein the gelled layer has a thickness of about 2 to about 5 mm, or about 2 to about 3 mm.
22. The gelled liquid test sample of any one of clauses 19-21, wherein the amount of glucomannan is from about 4% to about 10% (wt./wt.), about 5% to about 10% (wt./wt.), or from about 5% to about 7% (wt./wt.) of the liquid test sample.
23. The gelled liquid test sample of any one of clauses 19-22, wherein, in addition to the glucomannan, the gelling agent component comprises at least one of a gum, a natural polymer, and/or synthetic polymer.
24. The gelled liquid test sample of clause 23, wherein the gum comprises guar gum and/or xanthan gum.
25. The gelled liquid test sample of any one of clauses 19-24, wherein, prior to dissolution, the glucomannan of the glucomannan-containing gelling agent has been heat cured at a temperature and for a time sufficient to remove or substantially remove water therefrom and/or provide adequate sterilization thereof.
26. The gelled liquid test sample of any one of clauses 19-25, wherein the gelled layer spontaneously softens within a softening period of from about 2 to about 10 days, from about 2 to about 5 days, from about 3 to about 5 days, or within about 5 days, in each case measured from an initial gelling point.
27. The gelled liquid test sample of any one of clauses 19-26, further comprising a transparent container, the gelled layer of the liquid test sample being disposed therein.
28. A kit or combination of components for enumerating a target organism, comprising: a solid nutrient base media component; and a solid gelling agent component comprising glucomannan, wherein the amount of glucomannan is from about 45% to about 65% (wt./wt.) of the base media and the gelling agent components taken together.
29. The kit or combination of clause 28, wherein the amount of glucomannan is from about 50% to about 65% (wt./wt.) of the base media and the gelling agent components taken together.
30. The kit or combination of clause 28 or 29, wherein the amount of glucomannan is from about 4% to about 10% (wt./wt.), from about 5% to about 10% (wt./wt.), or from about 5% to about 7% (wt./wt.) of the liquid test sample testable by the kit.
31. The kit or combination of any one of clauses 28-30, wherein, in addition to the glucomannan, the gelling agent component comprises at least one of a gum, a natural polymer, and/or synthetic polymer.
32. The kit or combination of clause 31, wherein the gum comprises guar gum and/or xanthan gum.
33. The kit or combination of any one of clauses 28-32, wherein the glucomannan of the glucomannan-containing solid gelling agent component has been heat cured at a temperature and for a time sufficient to remove or substantially remove water therefrom and/or provide adequate sterilization thereof.
34. The kit or combination of any one of clauses 28-33, wherein the base media component and the solid gelling agent component are present as separate components of the kit or combination, or are present in the form of a mixed component in the kit or combination.
35. The kit or combination of any one of clauses 28-34, further comprising an antibiotic, and wherein the antibiotic in the kit or combination has a shelf-life stability of at least 0.9 years at room temperature, and/or at least 3.7 years at 5° C.
36. The kit or combination of clause 35, wherein the antibiotic comprises one or more of Vancomycin, Ciprofloxacin, Cefotaxime, and/or Methicillin.
37. The kit or combination of any one of clauses 28-36, further comprising a transparent testing container.
38. The kit of clause 37, wherein one or more inner surface(s) of the container is pre-coated with the solid cold water-soluble gelling component and/or the solid nutrient base media component.
39. A method for forming a uniform gel in a test sample container, comprising: retaining, on one or more inner surface(s) of a test sample container, a coating comprising a solid nutrient base media component and/or a solid glucomannan-containing gelling agent component of a gelable, solid cold water-soluble microbiological medium; introducing a liquid test sample into the sample container to dissolve the microbiological medium; and forming, in the container, a gelled layer of the dissolved microbiological medium, with no requirement for filtering or concentrating the liquid test sample.
40. The method of clause 39, wherein both the solid nutrient base media component and the solid gelling agent component are coated on the one or more inner surface(s) of the sample container.
41. The method of clause 39 or 40, wherein solid gelling agent component comprises glucomannan derived from Konjac root.
42. The method of any one of clauses 39-41, wherein the amount of glucomannan is from about 45% to about 65% (wt./wt.) of the base media and the gelling agent components taken together.
43. The method of clause 42, wherein the amount of glucomannan is from about 50% to about 65% (wt./wt.) of the base media and the gelling agent components taken together.
44. The method of any one of clauses 39-43, wherein the amount of glucomannan is from about 4% to about 10% (wt./wt.), from about 5% to about 10% (wt./wt.), or from about 5% to about 7% (wt./wt.) of the liquid test sample.
45. The method of any one of clauses 39-44, wherein, in addition to the glucomannan, the gelling agent component comprises at least one of a gum, a natural polymer, and/or synthetic polymer.
46. The method of clause 45, wherein the gum comprises guar gum and/or xanthan gum.
47. The method of any one of clauses 39-46, wherein the glucomannan has been heat cured at a temperature and for a time sufficient to remove or substantially remove water therefrom and/or provide adequate sterilization thereof.
48. The method of any one of clauses 39-47, wherein the dissolution and forming are at one or more temperature(s) in a range of 10° C. (50° F.) to 45° C. (113° F.).
49. The method of clause 48, wherein the dissolution and forming are at room temperature(s) or ambient temperature(s) in a range of about 16° C. (61° F.) and about 25° C. (77° F.).
50. The method of any one of clauses 39-49, as part of a method for enumerating a target organism in the test sample, further comprising: incubating the gelled layer at one or more incubation temperature(s) and for a time sufficient to provide for microbial growth and detectable microbiological colony formation in or on the gelled medium layer; and counting the colonies to enumerate a number of target microorganisms in the test sample, wherein less than 1 target organism/mL of test sample may be detected and enumerated.
51. A kit or combination of components for enumerating a target organism, comprising: a transparent test sample container; a solid nutrient base media component; and a solid glucomannan-containing gelling agent component, wherein the solid nutrient base media component and/or the solid gelling agent component is coated on one or more inner surface(s) of the test sample container.
52. The kit or combination of clause 51, wherein both the solid nutrient base media component and the solid gelling agent component are coated on the one or more inner surface(s) of the sample container.
53. The kit or combination of clause 51 or 52, wherein the solid gelling agent component comprises glucomannan derived from Konjac root.
54. The kit or combination of any one of clauses 51-53, wherein the amount of glucomannan is from about 45% to about 65% (wt./wt.) of the base media and the gelling agent components taken together.
55. The kit or combination of clause 54, wherein the amount of glucomannan is from about 50% to about 65% (wt./wt.) of the base media and the gelling agent components taken together.
56. The kit or combination of any one of clauses 51-55, wherein the amount of glucomannan is from about 4% to about 10% (wt./wt.), from about 5% to about 10% (wt./wt.), or from about 5% to about 7% (wt./wt.) of a liquid test sample testable by the kit.
57. The kit or combination of any one of clauses 51-56, wherein, in addition to the glucomannan, the gelling agent component comprises at least one of a gum, a natural polymer, and/or synthetic polymer.
58. The kit or combination of clause 57, wherein the gum comprises guar gum and/or xanthan gum.
59. The kit or combination of any one of clauses 51-58, wherein the glucomannan has been heat cured at a temperature and for a time sufficient to remove or substantially remove water therefrom and/or provide adequate sterilization thereof.

### BRIEF DESCRIPTION OF THE DRAWINGS

Those of skill in the art will understand that the drawing(s), described below, are for illustrative purposes only. The drawings are not intended to limit the scope of the present teachings in any way.

FIG. 1 shows, by way of non-limiting examples of the present invention, a photograph showing a test bag containing and displaying a gelled, cold water-soluble microbiological medium formed with 100 mL of test water in accordance with the present invention (Example 1). The colonies, blue-colored in reality, are E. coli.

### DETAILED DESCRIPTION OF THE INVENTION

Aspects of the invention overcome deficiencies of current methods in enumerating a low number of target organisms in water, beverage, and food samples by providing a rapid and easy-to-use enumeration method which allows enumerating as low as 1 cell of the target organisms in a relatively large volume (e.g., 5 ml, 10 mL, 50 ml, 100 mL, 150 ml, 500 ml, or 1000 mL, etc.) of test sample.

Aspects of the invention provide compositions comprising cold water-soluble gelling agent(s), and methods (e.g., enumerating bacteria, etc.) for using same. Provided, for example, are methods using one or more gelable cold water-soluble microbiological medium compositions, which comprise, e.g., a cold water-soluble gelling agent(s), a nutrient base medium, and optionally an indicator (e.g., color-developing) agent(s) and/or antibiotic(s), wherein the gelled compositions are suitable to enumerate a low level (e.g., less than 1 cell/mL) of target organisms in a high volume (e.g., 5 ml, 10 ml, 100 ml, 1000 ml, etc.) of a test sample. The disclosed gelable cold water-soluble microbiological media are used to gel (e.g., in the form of a layer having a suitable thickness and rigidity) a large volume (e.g., 5 ml, 10 mL, 50 ml, 100 mL, 150 ml, 500 ml, or 1000 mL, etc.), preferably 10 ml, 100 ml, or 1000 ml, of a test liquid sample at e.g., room temperature (e.g., 15°-25° C. (59°-77° F.) or suitable ambient temperature. The gelling may be, for example, within about 1-5 minutes, preferably within about 2-5 minutes, and most preferably within about 3-5 minutes after mixing the liquid test sample with the gelable cold water-soluble microbiological medium composition (e.g., in a suitably sized test sample bag, e.g., transparent plastic bag). The sealed sample bag containing the gelled layer is then incubated at, for example, either 37° C. for 24 h or ambient or room temperature for up to 72 h to allow the target organisms to grow in colonies (e.g., with specific colony colors; depending on the indicator used) within the gel. The methods provide for direct enumeration by a simple counting of the colonies observed, preferably using the unaided human eye. Additionally, the disclosed gels spontaneously soften and begin to liquify after a few days (e.g., preferably after about 5 days at, for example, ambient, room, or typical microbiological incubation temperatures), which substantially facilitates efficient and cost-effective disinfection and disposal.

### Advantages of the Disclosed Gels and Methods

The disclosed compositions and methods overcome several substantial disadvantages of art-recognized methods and gels used for microbiological enumeration, particularly in the context of enumerating microorganisms in large volume samples. For example, comparative MPN-based and membrane filter-based methods traditionally used for large (e.g., 10 mL, 100 mL or 1000 mL, etc.) samples having a low number of a target organism (e.g., less than 1 microorganism/mL) are either not precise (MPN; comparative) and/or are expensive and/or time consuming and may not be applicable to turbid samples. Additionally, particular current gel systems (e.g., pectin-based, etc.) require addition of divalent cations (e.g., calcium) for gelling, which additions may be undesirable or incompatible with desired target microbe growth. Other methods use gel agents that require heating for adequately dissolution, which heating not only adds time to the process, but also may not be compatible with heat tolerance of the target microorganism(s). Moreover, many such gel materials tend not to uniformly dissolve upon heating and have too many clumps, and/or the colony quality is not sufficiently distinct to provide for facile and accurate enumeration. Furthermore, once used for enumeration, such contaminated gelled materials persist in the gelled state for long periods (e.g., weeks, or longer) requiring work-intensive/inefficient and/or costly sterilization methods (e.g., autoclaving, etc.) to enable safe disposal of accumulated spent assay materials.

By contrast, the disclosed gelable cold water-soluble microbiological medium compositions and methods provide one or more substantial advantages over such art-recognized gels.

First, using the disclosed methods, large samples are readily processed by incorporating an appropriate amount of gelling agent, base media, etc., into the sample, and using an appropriately sized sample bag.

Second, because the disclosed cold-water gelling agents readily dissolve and uniformly gel in aqueous solution at room or typical ambient temperatures, there is no need for heating the gel material in dissolving or forming the gels for enumeration assays.

Third, there is no requirement for divalent cation addition for forming the gels for enumeration assays.

Fourth, the gelling agent(s) are cold water-soluble and form adequate gels in minutes, e.g., 2-5 minutes, preferably 3-5 minutes, providing for a rapid assay format, yet with adequate time for mixing of the media components and sample prior to forming the gelled layer within the assay container (e.g., test sample bag).

Fifth, the disclosed cold water-soluble microbiological medium compositions uniformly dissolve at room or moderate ambient temperatures without deleterious clumping, and the colony quality is sufficiently distinct to provide for facile and accurate enumeration. This is also true of the media-coated container embodiments described herein below, wherein one or more inner surface(s) of the test sample containers (e.g., transparent bags) are coated or lined (e.g., pre-coated, adhered) with a desired known amount of solid (e.g., dry powder, etc.) cold water soluble microbiological medium components (e.g., containing cold water soluble gelling agent(s) and/or a nutrient base medium). Such media-coated container embodiments not only (i) provide increased efficiency by eliminating the need to measure and add the solid cold water-soluble microbiological medium to the test container at the time of sample addition; but also (ii) further reduce or eliminate any initial solid media clumping upon contact with the liquid test sample, thereby providing further enhanced gel uniformity and reproducibility.

Sixth, in particular embodiments, the disclosed gelled layers spontaneously begin to soften and liquify after a few days (e.g., after about 2 to 5 days, after about 3 to 5 days, preferably after about 5 days at, for example, ambient, room, or typical microbiological incubation temperatures). Such softening and liquification enables, faster, more cost effective, and more efficient disinfection of used, contaminated gels (e.g., by addition of chlorine bleach, peroxide, etc. to the liquified material) prior to disposal.

### Components of the Gelable Medium

“Gelable, solid cold water-soluble microbiological medium” or “gelable cold water-soluble medium,” as used herein, generally refers to a medium (e.g., solid, powdered) comprising one or more cold water-soluble gelling agents, a nutrient base media, and optionally one or more indicators (e.g., color, fluorescent, etc.) and/or antibiotic(s). The cold water-soluble gelling agent(s) may, for example, be combined with or be in combination with the nutrient base medium prior to its introduction (dissolution, e.g., at room or ambient temperature) into, or mixing with, a liquid test sample, or may be introduced into, or mixed with, the liquid test sample before, or preferable after dissolution of the base medium into the liquid test sample.

Preferably, the liquid test sample is initially mixed with the nutrient base media (e.g., in a test sample bag, a sample container, or other suitable container) to facilitate initial dissolution of the nutrient base media in the sample, prior to introducing the cold water-soluble gelling agent(s) to the sample/nutrient base media mixture. This sequence allows more time for dissolution of the nutrient base medium prior to gel formation, thus helping to avoid clumping and/or non-uniformity of the resulting gelled microbiological medium.

Alternatively, if dissolution of the nutrient base media in the text sample is sufficiently rapid, then the nutrient base media and the cold water-soluble gelling agent(s) may be introduced (or mixed with) together (e.g., as a combination), or serially, in either order, in rapid succession (e.g., as two separate components), to the test sample.

In either case, the gelable cold water-soluble medium dissolves and forms a gelled microbiological medium in a few minutes (e.g., within about 1-5 minutes, more preferably about 2-5 minutes, most preferably about 3-5 minutes in a suitably sized test sample container/bag, e.g., transparent plastic bag).

Exemplary cold water-soluble gelling agents include but are not limited to one or more of guar gum, xanthan gum, glucomannan, locust bean gum, tica gum, carrageenan, alginate, gelatin, silica gel, methyl cellulose, etc.

The water-soluble gelling agents are present in the final test sample mixture (gelable, solid cold water-soluble gelling agent(s), plus nutrient base media, plus test sample) in an amount effective to provide sufficient gelling with a desired rigidity. Gel rigidity is preferably sufficient so that the gelled sheets (e.g., having a thickness of 2-5 mm, preferably 2-3 mm) do not collapse when the sample bags containing them are held vertically, and yet allow for an average colony size of about 1-2 mm, depending on the target organism.

The nutrient base media can be one or more of any culture media known in the art for growing microorganisms. The nutrient base media, for example, may comprise one or more of amino-nitrogen sources, growth factors, energy sources, buffer salts, mineral salts and selective growth agents that support the selective growth of indicator organisms, surrogate organisms or pathogens. Growth inhibitors may be included (e.g., bile salts to inhibits gram positives).

Exemplary specific nutrient base media examples, include but are not limited to one or more of: Modified mTEC, Baird-Parker medium, MSRV, Enterococcus (mEI medium), etc., as shown in Tables 1-4 below (* indicates presence as an inhibitor of non-target bacteria; and note that use of sodium lauryl sulfate in Tables 1 and 3 is optional).

TABLE 1
Modified mTEC (e.g., for E. coli and total coliforms)

| Ingredient | Wt. in gram |
|---|---|
| Peptone special | 5 |
| Sodium Chloride | 7.5 |
| K₂HPO₄ | 3.3 |
| KH₂PO₄ | 1 |
| Yeast extract | 3 |
| Sodium lauryl sulfate* | 0.2 |
| Sodium Desoxycholate* | 0.1 |
| IPTG | 0.15 |
| X-Gluc | 0.175 |
| Red Gal | 0.25 |
| Antifoam powder | 0.5 |

TABLE 2
Baird-Parker medium

| Ingredient | Wt. in gram |
|---|---|
| Bacto Tryptone | 10 |
| Bacto beef ext | 5 |
| Bact yeast | 1 |
| Lithium Chloride* | 5 |
| Glycine | 12 |
| Sodium pyruvate | 10 |
| Potassium Tellurite* | 0.1 |
| Sodium carbonate | 0.1 |
| Dry egg yolk | 12 |
| IPTG | 0.15 |
| Salmon-a-Gluc | 0.15 |
| Antifoam | 0.5 |

TABLE 3
MSRV

| Ingredient | Wt. in gram |
|---|---|
| Tryptone | 2.3 |
| Sodium Chloride | 7.34 |
| MgCl2 | 10.9 |
| KH2PO4 | 1.5 |
| Enzyme digest casein | 2.3 |
| Casein acid hydrolysate | 4.65 |
| Sodium lauryl sulfate* | 0.1 |
| (optional ingredient) | |
| Novobiocin* | 0.01 |
| IPTG | 0.15 |
| X-a-Gal | 0.15 |
| Red Gal | 0.2 |
| Antifoam powder | 0.5 |

TABLE 4
Eneterococcus (mEI medium)

| Ingredient | Wt. in gram |
|---|---|
| Peptone | 10 |
| Sodium Chloride | 15 |
| Cycloheximide* | 0.05 |
| Sodium azide* | 0.15 |
| Yeast extract | 30 |
| 3-indoxy-b-D- | 0.75 |
| glucoside | |
| IPTG | 0.15 |

Exemplary indicator color-developing agents include but are not limited to one or more of pH indicators, triphenyltetrazolium chloride, 3-indoxy-b-D-glucoside (IBDG), X-Gluc (5-bromo-4-chloro-3-indolyl-beta-D-glucuronic acid, cyclohexylammonium salt), Red-Gal (Rose-Gal, 6-Chloro-3-indolyl-β-D-galactopyranoside; Salmon-Gal), Salmon-a-Gluc (6-Chloro-3-indolyl-beta-D-glucuronic acid cyclohexylammonium salt), X-α-Gal (5-Bromo-4-chloro-3-indoxyl-α-D-galactopyranoside), or other suitable chromogenic substrates (e.g., that are hydrolysed by the bacterial enzymes such as phosphatase, glucosidase, glucuronidase, galactosidase, proteases and esterases), etc. Fluorescent indicators (U.V. detectable) may be employed. The indicator(s) may be added to the test sample, or to the test sample mixture as an independent component, or in combination with either the gelling agent(s) and/or nutrient base medium components.

Exemplary antibiotics include but are not limited to one or more of Cefotaxime, Methicillin, Vancomycin, Ciprofloxacin, Novobiocin, etc.

### Exemplary Enumeration Procedures:

In cases where dissolution of the selected nutrient base media in the liquid test sample is sufficiently rapid, such that the nutrient base media and the cold water-soluble gelling agent(s) may be introduced together (e.g., as a combination), or serially in either order in rapid succession (e.g., as two separate components added concurrently or substantially so), to the test sample, then a specified volume of aqueous sample may be introduced (e.g., poured) into an appropriately sized container (e.g., plastic bag) containing a cold water soluble microbiological medium (cold water soluble gelling agent(s), plus the nutrient base medium, optionally plus indicator(s) and/or antibiotic(s)); that is:
1. Pour a specified volume of an aqueous test sample into a specially sized plastic bag containing a cold water-soluble microbiological medium. In a preferred embodiment, the bag (e.g., 7×8 inch (17.8×20.3 cm)) is capable of holding 100 mL of the aqueous sample and a specific quantity of the cold water-soluble microbiological medium mixture;
2. Gently mix by hand to mix and dissolve the gelable cold water-soluble microbiological medium into the liquid sample, and flatten the contents of the bag into a layer (e.g., having a thickness of 2-5 mm, preferably 2-3 mm) using a scraper, roller, squeegee, or other suitable device while displacing any air pockets (e.g., bubbles) to provide a uniform or substantially uniform gelable layer within the bag; initial flattening of the contents in the bag while supported at a slight incline towards the upper open bag end, facilitates displacement of air from the gel layer, and optionally from the bag itself, exiting at the higher inclined open end;
3. Seal the plastic bag, allow the gelable mixture to gel, and incubate at 37° C. for 24 h or room temperature for up to 72 h (or other suitable temperature and time, e.g., 35° C., 44.5° C., etc.) (alternatively, the bag may be sealed and then flattened prior to gelling); and
4. Count the target colonies (e.g., having specific indicator color(s)).

Alternatively, the cold water soluble microbiological medium (cold water-soluble gelling agent(s) plus the nutrient base medium), may be added to an appropriately sized container (e.g., plastic bag) containing the aqueous test sample, followed by mixing, sealing the container/bag, incubating and counting the resulting colonies.

Preferably, as mentioned above, and particularly in cases where dissolution of the selected nutrient base media in the liquid test sample may not be sufficiently rapid to achieve adequate or complete dissolution prior to onset of gelling of the cold water-soluble gelling agent(s), the liquid test sample may initially be mixed with the nutrient base media (e.g., in a test sample bag, a sample container, or other suitable container) to facilitate initial dissolution of the nutrient base media in the aqueous sample, prior to introducing the cold water-soluble gelling agent(s) to the sample/nutrient base media mixture, thereby allowing more time for dissolution of the nutrient base medium prior to gel formation, thus helping to avoid clumping and/or non-uniformity of the resulting gelled microbiological medium; that is:
1. Pour a specified volume of an aqueous test sample into a specially sized plastic bag containing a selected nutrient base media (alternately, the nutrient base media may be added to a bag containing the sample). In a preferred embodiment, the bag (e.g., 7×8 inch (17.8×20.3 cm)) is capable of holding 100 mL of the aqueous sample and a specific quantity of the final cold water-soluble microbiological medium mixture;
2. Gently mix by hand to dissolve the nutrient base media into the aqueous sample;
3. Introduce the cold water-soluble gelling agent(s) to the sample/nutrient base media mixture;
4. Gently mix by hand to mix and dissolve the cold water-soluble gelling agent(s) into the nutrient base media/aqueous sample mixture, and flatten the contents of the bag into a layer (e.g., having a thickness of 2-5 mm, preferably 2-3 mm) using a scraper, roller, squeegee, or other suitable device while displacing any air pockets (e.g., bubbles) to provide a uniform or substantially uniform gelable layer within the bag; initial flattening of the contents in the bag while supported at a slight incline towards the upper open bag end, facilitates displacement of air from the gel layer, and optionally from the bag itself, exiting at the higher inclined open end;
5. Seal the plastic bag, allow the gelable mixture to gel, and incubate at 37° C. for 24 h or room temperature for up to 72 h (or other suitable temperature and time, e.g., 35° C., 44.5° C., etc.) (alternatively, the bag may be sealed and then flattened prior to gelling); and
6. Count the target colonies (e.g., with specific color(s)).

Alternatively, the cold water soluble microbiological medium (cold water-soluble gelling agent(s) plus the nutrient base medium), may be added to an appropriately sized container (e.g., plastic bag) containing the aqueous test sample, followed by mixing, sealing the container/bag, incubating and counting the resulting colonies.

### Media-Coated Container Embodiments.

Alternatively, the test gels may be formed by introducing a liquid test sample into a suitably sized test sample container (e.g., transparent plastic bag) having one or more inner surface(s) coated or lined (e.g., pre-coated, adhered layer) with a selected/known amount of solid (e.g., dry powder, etc.) cold water soluble microbiological medium or component thereof (e.g., containing cold water soluble gelling agent(s) and/or a nutrient base medium), and allowing the coated solid pre-coated cold water soluble microbiological medium or component thereof to dissolve and gel in the liquid test sample within the container (e.g., bag). Such media-lined container embodiments not only (i) provide increased efficiency by eliminating the need to measure and add the solid cold water-soluble microbiological medium or component thereof to the test container; but also (ii) help further reduce or eliminate any initial solid media (or component thereof) clumping upon contact with the liquid test sample, thereby providing further enhanced gel uniformity and reproducibility. Either, or both of the cold-water soluble gelling agent(s) component and/or the nutrient base medium component may be coated on one or more inner surfaces of the test sample container. The cold-water soluble microbiological medium components may be mixed together and coated as/within one or more layers, or the components may be coated in separate respective layers over the same or different inner surface area(s) of test sample container. Non-limiting examples include:
1. Pour a specified volume of an aqueous test sample into a specially sized plastic bag having an internal surface(s) coated with a solid cold water-soluble microbiological medium (gelling component plus nutrient base medium component). In a preferred embodiment, the bag (e.g., 7×8 inch (17.8×20.3 cm)) is capable of holding 100 mL of the aqueous sample and one or more areas of its internal surface is pre-coated (e.g., by use of an adhesive agent) with a specific known quantity (e.g., 9.61 g as in Table 5 below) of the solid cold water-soluble microbiological medium (e.g., containing cold water soluble gelling agent(s) and a nutrient base medium);
2. Gently mix by hand to mix and dissolve the gelable cold water-soluble microbiological medium into the liquid sample, and flatten the contents of the bag into a layer (e.g., having a thickness of 2-5 mm, preferably 2-3 mm) using a scraper, roller, squeegee, or other suitable device while displacing any air pockets (e.g., bubbles) to provide a uniform or substantially uniform gelable layer within the bag; initial flattening of the contents in the bag while supported at a slight incline towards the upper open bag end, facilitates displacement of air from the gel layer, and optionally from the bag itself, exiting at the higher inclined open end;
3. Seal the plastic bag, allow the gelable mixture to gel, and incubate at 37° C. for 24 h or room temperature for up to 72 h (or other suitable temperature and time, e.g., 35° C., 44.5° C., etc.) (alternatively, the bag may be sealed and then flattened prior to gelling); and
4. Count the target colonies (e.g., having specific indicator color(s)).

Such media-coated/lined container embodiments are preferably used in cases where dissolution of the selected nutrient base media in the liquid test sample is sufficiently rapid, such that the nutrient base media and the cold water-soluble gelling agent(s) may be introduced together (e.g., as a solid combination, or as separate respect layers, adhered to one or more inner surfaces of the sample container) in the liquid test sample.

In alternate media-coated container embodiments, the test gels may be formed by introducing a liquid test sample, containing either (i) a solid (e.g., dry powder, etc.) cold water soluble gelling agent(s) component or (ii) a solid nutrient base medium component, into a suitably sized test sample container (e.g., transparent plastic bag) having one or more inner surface(s) coated or lined (e.g., pre-coated, adhered) with the complementary media component (the a solid nutrient base medium or solid cold water soluble gelling agent(s) component, respectively, and allowing the solid pre-coated component to dissolve and the mixture gel within the container (e.g., bag). Such media-coated container embodiments not only (i) provide increased efficiency by eliminating the need to measure and add one or more solid components of the cold water-soluble microbiological medium to the test container; but also (ii) reduce or eliminate any initial solid media clumping upon contact with the liquid test sample, thereby providing further enhanced gel uniformity and reproducibility.

In the above-described media-coated container embodiments, the containers (e.g., bags) may, for example, be charged/lined/coated with one or both of the solid component(s) of the cold water-soluble microbiological medium (e.g., the gelling agent and/or the nutrient base media) using any suitable adhesive or coating technique. For example, one or more solid components (e.g., in powder form) of a solid cold water-soluble microbiological medium may be applied to and/or adhered by a suitable adhesive. The adhesive may form an aggregate or mixture with the dry components of the microbiological medium, and/or form a layer under or over the dry components of the microbiological medium, in any case retaining the dry components of the microbiological medium as a coating or layer on one or more inner surfaces of the test sample container.

Suitable adhesives may, for example, include acrylate-based adhesives, such as water-insoluble isooxtyl acrylate adhesives, alkyl acrylate/acrylic acid adhesive, isooctyl acrylate/acrylamide (e.g., in a weight ratio of 96/4), isooctyl acrylate/acrylic acid (see e.g., U.S. Pat. No. 5,409,838). Other suitable exemplary adhesive may include a water insoluble adhesive comprising a copolymer of an alkyl acrylate monomer and alkyl amide monomer (e.g., in a ratio from 90:10-99:1, more preferably 94:6 to 98:2). The alkyl acrylate monomer may, for example, comprise a lower alkyl (C2-C10) monomer of acrylic acid, including, for example, isooctyl acrylate (IOA), 2-ethylhexyl acrylate, butyl acrylate, ethyl acrylate, isoamyl acrylate, and mixtures thereof, while the alkyl amide monomer may, for example, comprise, without limitation, acrylamide (ACM), methacrylamide, N-vinylpyrrolidone (NV), N-vinylcaprolactam (NVCL), N-viny-2-piperidine, N-(mono- or di-lower alkyl (C2 to C5)) (meth) acrylamides, N-methyl (meth) acrylamide, N, N dimethyl (meth) acrylamides, or mixtures thereof (see, e.g, U.S. patent application Ser. No. 16/977,244 (published as US 2021/0040434). Alternatively, polysaccharide-based adhesives may be used.

In particular embodiments, the test sample containers are transparent plastic (e.g., polyethylene, etc.) bags formed by sealing together opposed sheets of clear plastic film (e.g., sealing two separate sheets, or sealing opposing portions of a folded sheet), wherein at least one of the inner opposed surface(s) is charged/lined/coated with at least one component of the cold water-soluble microbiological medium (e.g., a solid cold water soluble gelling agent(s) and/or a solid nutrient base medium). In manufacturing of the test sample containers, the opposing sheets may be derived, for example, from one or more rolls of plastic sheeting material (e.g., stock polyethylene rolls), wherein one side of the film of at least one roll is charged/lined/coated with at least one solid component of the cold water-soluble microbiological medium, prior to sealing of the opposed sheets to form the bag. The charging/lining/coating of the plastic sheeting material may take place in forming of the stock roll(s), or after forming the stock roll(s), but prior to sealing the opposed sheets during formation of the container bags.

### Definitions (Unless Otherwise Indicated)

“Target organisms” or “target microorganism,” as used herein, generally refers to any indicator organisms, surrogate organisms, or pathogens in, e.g., water, beverage and food samples.

“A large volume of test sample” refers to any volumes that are equal to or larger than 1 mL (e.g., >1 ml, 5 ml, 10 ml, 50 ml, 100 ml, 500 ml, 1000 ml, etc., or any intermediate volume, etc.). In particular aspects, the test sample is a volume in the range of from about 50 ml to about 150 ml. In certain aspects, the test sample is a volume of about 100 ml.

A “liquid test sample,” as used herein, refers to a test sample in liquid form. For example, the liquid test sample may be inherently in liquid form (e.g., an aqueous environmental sample, etc.), or may be a non-liquid sample (e.g., solid or semi-solid) that is dissolved or solubilized to be in liquid form.

“Test sample bag” or “sample bag,” as used herein, generally refers to bag suitably sized to contain a respective test sample volume (e.g., 5 ml, 10 mL, 100 mL or 1000 mL, etc.) and a cold water-soluble microbiological medium comprising a cold water-soluble gelling agent(s), in a gelled sheet, preferably having a uniform thickness of about 2-5 mm (preferably 2-3 mm), and where the bag can be made of any suitable transparent material(s). For example, for test sample volumes of 10 ml, 100 ml, and 1000 ml, 5×5 inch (12.7×12.7 cm), 7×8 inch (17.8×20.3 cm), and 9×12 inch (22.9×30.5 cm) bags, respectively, provide for uniform gelled media sheets of about 2-3 mm thickness. Colors are, however, still visible up to about 0.5 cm gelled thickness.

“Cold water soluble,” as used herein, generally refers to being soluble in water at a temperature in the range of 10° C. (50° F.) to 45° C. (113° F.), preferably a temperature(s) selected so as not to adversely affect the survival of, e.g., a heat-sensitive target microorganism.

“Gelable, solid cold water-soluble microbiological medium” or “gelable cold water-soluble medium,” as used herein, generally refers to a solid (e.g., powdered) medium formed by combining one or more water-soluble gelling agents, a nutrient base media, and one or more indicators (e.g., color, fluorescent, etc.) and/or antibiotic(s). Exemplary cold water-soluble gelling agents include but are not limited to one or more of guar gum, xanthan gum, glucomannan, locust bean gum, tica gum, carrageenan, alginate, gelatin, silica gel, methyl cellulose, etc. The water-soluble gelling agents are present in the growth medium in an amount effective to provide sufficient gelling with a desired rigidity. Gel rigidity is preferably sufficient so that the gelled sheets (e.g., having a thickness of 2-5 mm, preferably 2-3 mm) do not collapse when the sample bags containing them are held vertically, and yet allow for an average colony size of about 1-2 mm, depending on the target organism. The nutrient base media can be one or more of any culture media known in the art for growing microorganisms. The nutrient base media may comprise one or more of amino-nitrogen sources, growth factors, energy sources, buffer salts, mineral salts and selective growth agents that support the selective growth of indicator organisms, surrogate organisms or pathogens. Exemplary nutrient base media are described above, and include but are not limited to one or more of: Modified mTEC, Baird-Parker medium, MSRV, Enterococcus (mEI medium), etc.

The term “room temperature” refers, unless stated otherwise, to any temperature within a range of ambient temperatures between about 16° C. (approximately 61° F.) and about 25° C. (approximately 77° F.). Commonly, room temperature is between about 20° C. and 22° C. (68° F.-72° F.).

The term “glucomannan” as used herein refers, unless stated otherwise, to a polysaccharide heteroglycan consisting of beta-(1->4)-linked D-glucose (G) and D-mannose (M) in a proportion of about 5:8 (e.g., the basic polymeric repeating unit having the pattern: GGMMGMMMMMGGM), with branching through beta-(1→3)- and beta-(1→6)-glucosyl linkages. Acetate groups may be, and preferably are present on C-6 every 9-19 units of the main chain. A recognized and preferred source of glucomannan is the fiber extracted from the tuber or root of the elephant yam, also known as konjac (Amorphophallus konjac or Amorphophallus rivieri).

The term “substantially” as used herein is meant to convey that in certain aspects the methods may encompass some amount of variation, e.g., 10% or less, 9% or less, 8% or less, 7% or less, 6% or less, 5% or less, 4% or less, 3% or less, 2% or less, 1% or less.

The term “about” as used herein in relation to recited values is intended to convey, unless stated otherwise, that values within 10% of the recited value are encompassed.

### EXAMPLES

The following non-limiting working examples are provided to further illustrate particular embodiments of the invention disclosed herein.

### Example 1

(E. coli was Enumerated in a 100 mL Volume of Test Sample)

This example describes enumeration of E. coli in a 100 ml test sample volume, using an exemplary gelable cold water-soluble microbial medium according to aspects of the present invention.

Microbiological medium formulation. The microbiological test medium comprised guar gum, xanthan gum, glucomannan, nutrient base media (culture media) ingredients, and 5-bromo-4-chloro-3-indolyl-β-D-glucuronide, in the amounts specified in Table 5 below, which are sufficient to enumerate E. coli in 100 mL of a water sample.

TABLE 5
Gelable cold water-soluble microbiological
medium used for enumeration of E. Coli.

| | Amount in grams, for use with |
|---|---|
| Ingredients | 100 ml of test sample |
| glucomannan | 5.5 |
| Guar gum | 2 |
| Xanthan gum | 0.5 |
| Peptone # 3 | 1 |
| Sodium dihydrogen phosphate | 0.2 |
| Disodium hydrogen phosphate | 0.25 |
| Bile salts No. 3 | 0.15 |
| 5-bromo-4-chloro-3-indolyl-β-D- | 0.01 |
| glucuronide | |

Enumeration method. The enumeration method employed in this example, was as follows:
1. A 100 mL water test sample (at room or ambient temperature) was introduced into a transparent test container (i.e., clear plastic bag) of predetermined size containing 9.61 g of a gelable, cold water-soluble microbiological medium (see Table 5 above). The size of the bag was configured (7×8 inch (17.8×20.3 cm)) so as to be able to hold the gelled mixture of the 100 mL of water and the gelable cold-water-soluble microbiological medium in a gelled layer having a thickness of about of 2-5 mm, in this instance 2-3 mm.
2. The water sample and the gelable, cold water-soluble microbiological medium were gently mixed together by hand massaging the exterior of the bag to mix and dissolve the gelable medium in the test sample.
3. The plastic bag containing the dissolved gelable medium was then flattened and sealed (alternatively sealed and then flattened) to provide for a gelled layer, forming in 2-5 min, and having a thickness of 2-3 mm, followed by incubating sealed gelled layer at 37° C. for 24 h (alternatively, incubation at ambient or room temperature for up to 72 h, or another suitable combination of temperature or time, may be used).
4. Target E. coli colonies having specific indicator color(s) (in this instance blue) were then counted (directly on the gel, of from a color photograph of the gel) to provide for enumeration of a low level of target organisms (e.g., less than 1 per ml) in a high volume (e.g, 10, 100, 1000 ml, etc.) of test sample.

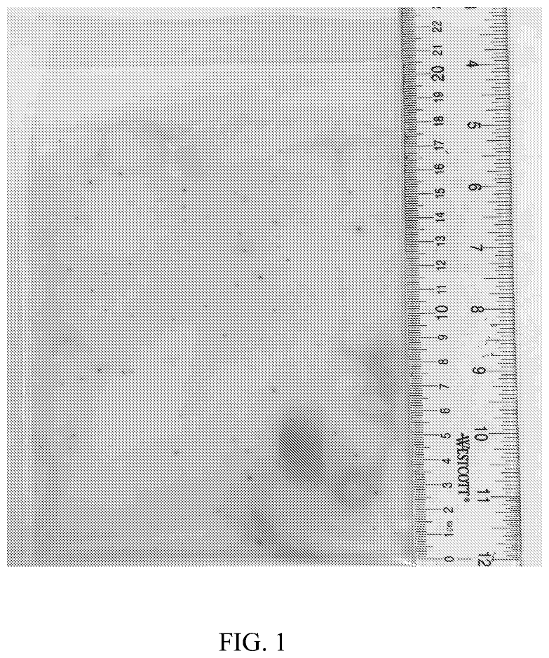

Results. FIG. 1 shows a photograph of the transparent test bag containing and displaying the gelled, cold water-soluble microbiological medium formed with the 100 mL test water sample. The colonies (blue-colored) are E. coli, and were counted by eye to enumerate the concentration of E. coli in the test sample.

As in this Example, the glucomannan employed in the gelable cold water-soluble microbiological medium is preferably that derived from konjac root (e.g., Amorphophallus konjac ground, processed and powdered such as that from Konjac; polymer; Nutrivita, CA; having particle size of 95% through 60 mesh). Preferably, prior to use in the disclosed gelable cold water-soluble microbiological media, the glucomannan powder is heat cured (e.g., baked in an oven) at one or more temperature(s) in a range of about 130° C. to about 200° C. or in any subrange thereof for at least about 1.5 h to about 2.5 h (e.g., at about 150° C. for at least about 2 h). According to aspects of the present invention, the inclusion of glucomannan (e.g., 5-7 g heat-cured glucomannan/100 ml sample) provides for: (i) enhanced uniformity of gelation of the dissolved gelable cold water-soluble medium, forming a uniformly gelled microbiological medium, at room or typical ambient temperatures, in a few minutes (e.g., within about 1-10 minutes, within about 1 to 5 min, more preferably within about 2-5 minutes, most preferably within about 3-5 minutes, yet providing adequate time for complete dissolution and enhanced uniformity of gelation for improved microbial enumeration; and additionally (ii) spontaneous softening and liquefaction after a few days (e.g., after about 2 to 10 days, after about 3 to 5 days, after about 4 to 5 days, preferably after about 5 days), enabling faster, more cost effective and efficient disinfection of used, contaminated gel material prior to disposal.

Gum, (e.g., guar gum, xanthan, etc.) is optionally added to fine-tune (e.g., extend) the duration of the gelled state (to maintain gel firmness longer if desired) prior to spontaneous softening/liquefaction of the gels, and thus can be used to further tailor the spontaneous softening/liquefaction timeline of the disclosed gelable cold water-soluble microbial media.

### Example 2

### (Total Coliform was Enumerated in a 100 mL Volume of Test Sample)

This example describes enumeration of total coliform in a 100 ml test sample volume, using a cold water-soluble gelling agent according to aspects of the present invention.

### Enumeration Method:

Microbiological medium formulation. The microbiological test medium comprised guar gum, xanthan gum, glucomannan, nutrient base media (culture media) ingredients, and 5-bromo-4-chloro-3-indolyl-β-D-galactopyranoside, in the amounts specified in Table 6 below, which are sufficient to enumerate total coliform in 100 mL of a test water sample.

TABLE 6
Gelable cold water-soluble microbiological medium
used for enumeration of total coliform.

| | Amount in grams, for use with |
|---|---|
| Ingredients | 100 ml of test sample |
| glucomannan | 5.5 |
| Guar gum | 2 |
| Xanthan gum | 0.5 |
| Peptone # 3 | 1 |
| Lactose | 1 |
| Sodium dihydrogen phosphate | 0.2 |
| Disodium hydrogen phosphate | 0.25 |
| Bile salts No. 3 | 0.15 |
| 5-bromo-4-chloro-3-indolyl-β-D- | 0.02 |
| galactopyranoside | |

Enumeration method. See enumeration method of Example 1, except that 10.62 g of a gelable, cold water-soluble microbiological medium was used (see formulation in Table 6 above).

Results. Total coliform was enumerated by counting colored colonies on the gel as in Example 1.

### Example 3

(Total Coliform and E. coli were Simultaneously Enumerated in a 100 mL Volume of Test Sample)

This example describes simultaneous enumeration of total coliform and E. coli in a 100 ml test sample volume, using a cold water-soluble gelling agent according to aspects of the present invention.

Microbiological medium formulation. The microbiological test medium comprised guar gum, xanthan gum, glucomannan, nutrient base media (culture media) ingredients, 5-bromo-4-chloro-3-indolyl-β-D-galactopyranoside and 6-chloro-3-indolyl-β-D-galactopyranoside, in the amounts specified in Table 7 below, which are sufficient to enumerate total coliform in 100 mL of a test water sample.

TABLE 7
Gelable cold water-soluble microbiological medium used
for enumeration of E. coli and total coliform.

| | Amount in grams, for use with |
|---|---|
| Ingredients | 100 ml of test sample |
| glucomannan | 5.5 |
| Guar gum | 2 |
| Xanthan gum | 0.5 |
| Peptone # 3 | 1 |
| Lactose | 1 |
| Sodium dihydrogen phosphate | 0.2 |
| Disodium hydrogen phosphate | 0.25 |
| Bile salts No. 3 | 0.15 |
| 5-bromo-4-chloro-3-indolyl-β-D- | 0.01 |
| glucuronide | |
| 6-Chloro-3-indolyl-β-D- | 0.02 |
| galactopyranoside | |

Enumeration method. See enumeration method of Example 1, except that 10.63 g of a gelable, cold water-soluble microbiological medium was used (see formulation in Table 6 above).

Results. E. coli and total coliform was enumerated by counting colored colonies (blue and red) on the gel as in Examples 1 and 2.

### Example 4

(Gelable, Solid Cold Water-Soluble Microbiological Media Containing Antibiotics were Shown to be Surprisingly Stable)

According to particular aspects of the present invention, applicant is the first to incorporate antibiotics (e.g., vancomycin, amoxicillin, etc.) into the base media component and/or the solid gelling agent component of a solid (e.g., powdered) gelable, cold water-soluble microbiological media) containing glucomannan, wherein the antibiotics are stable at room temperature for up to at least 0.9 years, and up to at least 3.7 years at 5° C., as shown by the shelf-life results in Table 1 below.

TABLE 1
Shelf life of the exemplary disclosed powered
medium with antibiotics as indicated

| | Shelf-life of antibiotic stability | |
|---|---|---|
| Powdered medium | At room temperature | With refrigeration |
| w/antibiotics | (25° C.) | (5° C.) |
| Modified mTEC/ | 336 days | 1344 days |
| Cefotaxime | | |
| Baird-Parker-Methicillin | 336 days | 1344 days |
| Baird-Parker-Vancomycin | 336 days | 1344 days |
| MSRV/Ciprofloxacin | 336 days | 1344 days |

Preferably, the antibiotic is part of a kit or combination, wherein the antibiotic in the kit or combination has a shelf-life stability of at least 0.9 years at room temperature, and/or at least 3.7 years at 5° C.

### Example 5

### (Kits Comprising Gelable, Solid Cold Water-Soluble Microbiological Media Containing Antibiotics are Provided)

According to particular aspects of the present invention, kits for enumerating a target organism are provided wherein the kits comprise: a solid nutrient base media component as described and disclosed herein; and a solid gelling agent component comprising glucomannan, wherein the amount of glucomannan is from about 45% to about 65% wt./wt. of the base media and the gelling agent components taken together. Preferably, the amount of glucomannan is from about 50% to about 65% wt./wt. of the base media and the gelling agent components taken together. Most preferably the amount of glucomannan is from about 5 g to about 7 g per 100 ml of liquid test sample testable by the kit.

In the kits, the base media component and the solid gelling agent component may be present as separate components of the kit (e.g., contained in separate containers within the kit), so that they may combined, in use of the kit, serially or concurrently with a liquid test sample. Alternatively, the base media component and the solid gelling agent component may be present in the form of a mixed component of the kit (e.g., mixed together in a container within the kit) that may be concurrently combined, in use of the kit, to a liquid test sample.

The kits may optionally comprise a sample container that may already contain the base media component and/or the solid gelling agent component (e.g., either loose within the container, or coated on one of more internal container surfaces—such as the media-coated sample container embodiments disclosed herein), for mixing, in use of the kit, with a liquid test sample. The container is preferably transparent, and preferably sized to hold the base media component and/or the solid gelling agent component combined with a volume of liquid test sample in a gelled layer. For example, for test sample volumes of 10 ml, 100 ml, and 1000 ml, 5×5 inch (12.7×12.7 cm), 7×8 inch (17.8×20.3 cm), and 9×12 inch (22.9×30.5 cm) bags, respectively, provide for uniform gelled media sheets of about 2-3 mm thickness. Colors (e.g., colorimetric or fluorescent marker) for colony visualization on or within the gelled layer are, however, still visible in gelled layers up to about 0.5 cm gelled thickness.

In the kits, the gelling agent component may comprise a gum (e.g., wherein the gum may comprises guar gum and/or xanthan gum.

The kits may further comprise one or more antibiotic(s) (e.g., Vancomycin, Ciprofloxacin, Cefotaxime, Methicillin, etc.).

The antibiotic, when formulated with the base media component and/or the solid gelling agent component in the kits, has a shelf-life stability at room temperature for up to 0.9 years or longer, and up to 3.7 years or longer at 5° C.

1. A method for enumerating a target organism in a test sample, comprising:
retaining, in a suitable container, a liquid test sample having dissolved therein a nutrient base media component and a glucomannan-containing gelling agent component of a gelable, solid cold water-soluble microbiological medium;
forming, in the container, a gelled layer of the dissolved medium within a gelling time period of less than 1 hour after dissolution of the gelling agent component in the liquid test sample;
incubating the gelled layer at one or more incubation temperature(s) and for a time sufficient to provide for microbial growth and detectable microbiological colony formation in or on the gelled medium layer; and
counting the colonies to enumerate a number of target microorganisms in the test sample, wherein less than 1 target organism/mL of test sample may be detected and enumerated, with no requirement for filtering or concentrating the liquid test sample.

2. The method of claim 1, wherein the test sample is a volume in the range of from about 50 ml to about 150 ml.

3. The method of claim 1 or 2, wherein the gelling time period is within about 1 to about 10 minutes, within about 1 to 5 minutes, within about 2 to about 5 minutes, or within about 3 to about 5 minutes, in each case after combining the gelling agent component with the liquid test sample.

4. The method of any one of claims 1-3, wherein the dissolution and forming are at one or more temperature(s) in a range of 10° C. (50° F.) to 45° C. (113° F.).

5. The method of any one of claims 1-4, wherein the amount of glucomannan is from about 4% to about 10% (wt./wt.), from about 5% to about 10% (wt./wt.), or from about 5% to about 7% (wt./wt.) of the liquid test sample.

6. The method of any one of claims 1-5, wherein, in addition to the glucomannan, the gelling agent component comprises at least one of a gum, a natural polymer, and/or synthetic polymer.

7. The method of claim 6, wherein the gum comprises guar gum and/or xanthan gum.

8. The method of any one of claims 1-7, wherein, prior to dissolution in the liquid test sample, the glucomannan of the glucomannan-containing gelling agent component of the gelable, solid cold water-soluble microbiological medium has been heat cured at a temperature and for a time sufficient to remove or substantially remove water therefrom and/or provide adequate sterilization thereof.

9. The method of any one of claims 1-8, wherein the dissolution and forming are at room temperature(s) or ambient temperature(s) in a range of about 16° C. (61° F.) and about 25° C. (77° F.).

10. The method of any one of claims 1-9, wherein the incubation temperature is equal to, or greater than, the dissolution and forming temperature.

11. The method of any one of claims 1-10, wherein the gelled layer has a thickness of about 1 to about 5 mm, about 2 to about 5 mm, or about 2 to about 3 mm, and/or wherein the container is transparent.

12. The method of any one of claims 1-11, wherein the gelled layer comprises a marker agent, and/or an antibiotic, and/or an inhibiting agent.

13. The method of claim 12, wherein the marker is a colorimetric or a fluorescent marker.

14. The method of any one of claims 1-13, wherein the nutrient base media component and the gelling agent component are separate components that are dissolved in the liquid test sample by dissolving them serially or concurrently, or wherein the nutrient base media component and the gelling agent component are in the form of a mixed component that is dissolved in the liquid test sample.

15. The method of any one of claims 1-13, wherein the nutrient base media component and the gelling agent component are separate components, and the dissolving of the nutrient base media component in the liquid test sample precedes the dissolving of the gelling agent component in the liquid test sample.

16. The method of claim 15, wherein the nutrient base media is a component of the liquid test sample.

17. The method of any one of claims 1-15, wherein an inner surface of the sample container is pre-coated with the solid cold water-soluble gelling agent(s) and/or the solid nutrient base medium, and retaining comprises initially introducing the liquid test sample into the pre-coated sample container.

18. The method of any one of claims 1-17, wherein after forming of the gelled layer, the formed gel layer spontaneously softens within a softening period of from about 2 to about 10 days, from about 2 to about 5 days, from about 3 to about 5 days, from about 4 to about 5 days, or within about 5 days.

19. A gelled liquid test sample, comprising a gelled layer of a liquid test sample containing a dissolved nutrient base medium and a dissolved glucomannan-containing gelling agent, wherein the gelled layer has a thickness of about 1 to about 5 mm, wherein the amount of glucomannan is from about 4% to about 10% (wt./wt.) of the liquid test sample, and wherein the gelled layer of the liquid test sample is suitable to provide for microbial growth and detectable microbiological colony formation therein and/or thereon.

20. The gelled liquid test sample of claim 19, wherein the gelled layer of the liquid test sample has a volume in the range of from about 50 ml to about 150 ml.

21. The gelled liquid test sample of claim 19 or 20, wherein the gelled layer has a thickness of about 2 to about 5 mm, or about 2 to about 3 mm.

22. The gelled liquid test sample of any one of claims 19-21, wherein the amount of glucomannan is from about 4% to about 10% (wt./wt.), about 5% to about 10% (wt./wt.), or from about 5% to about 7% (wt./wt.) of the liquid test sample.

23. The gelled liquid test sample of any one of claims 19-22, wherein, in addition to the glucomannan, the gelling agent component comprises at least one of a gum, a natural polymer, and/or synthetic polymer.

24. The gelled liquid test sample of claim 23, wherein the gum comprises guar gum and/or xanthan gum.

25. The gelled liquid test sample of any one of claims 19-24, wherein, prior to dissolution, the glucomannan of the glucomannan-containing gelling agent has been heat cured at a temperature and for a time sufficient to remove or substantially remove water therefrom and/or provide adequate sterilization thereof.

26. The gelled liquid test sample of any one of claims 19-25, wherein the gelled layer spontaneously softens within a softening period of from about 2 to about 10 days, from about 2 to about 5 days, from about 3 to about 5 days, or within about 5 days, in each case measured from an initial gelling point.

27. The gelled liquid test sample of any one of claims 19-26, further comprising a transparent container, the gelled layer of the liquid test sample being disposed therein.

28. A kit or combination of components for enumerating a target organism, comprising:
a solid nutrient base media component; and
a solid gelling agent component comprising glucomannan, wherein the amount of glucomannan is from about 45% to about 65% (wt./wt.) of the base media and the gelling agent components taken together.

29. The kit or combination of claim 28, wherein the amount of glucomannan is from about 50% to about 65% (wt./wt.) of the base media and the gelling agent components taken together.

30. The kit or combination of claim 28 or 29, wherein the amount of glucomannan is from about 4% to about 10% (wt./wt.), from about 5% to about 10% (wt./wt.), or from about 5% to about 7% (wt./wt.) of the liquid test sample testable by the kit.

31. The kit or combination of any one of claims 28-30, wherein, in addition to the glucomannan, the gelling agent component comprises at least one of a gum, a natural polymer, and/or synthetic polymer.

32. The kit or combination of claim 31, wherein the gum comprises guar gum and/or xanthan gum.

33. The kit or combination of any one of claims 28-32, wherein the glucomannan of the glucomannan-containing solid gelling agent component has been heat cured at a temperature and for a time sufficient to remove or substantially remove water therefrom and/or provide adequate sterilization thereof.

34. The kit or combination of any one of claims 28-33, wherein the base media component and the solid gelling agent component are present as separate components of the kit or combination, or are present in the form of a mixed component in the kit or combination.

35. The kit or combination of any one of claims 28-34, further comprising an antibiotic, and wherein the antibiotic in the kit or combination has a shelf-life stability of at least 0.9 years at room temperature, and/or at least 3.7 years at 5° C.

36. The kit or combination of claim 35, wherein the antibiotic comprises one or more of Vancomycin, Ciprofloxacin, Cefotaxime, and/or Methicillin.

37. The kit or combination of any one of claims 28-36, further comprising a transparent testing container.

38. The kit of claim 37, wherein one or more inner surface(s) of the container is pre-coated with the solid cold water-soluble gelling component and/or the solid nutrient base media component.

39. A method for forming a uniform gel in a test sample container, comprising:
retaining, on one or more inner surface(s) of a test sample container, a coating comprising a solid nutrient base media component and/or a solid glucomannan-containing gelling agent component of a gelable, solid cold water-soluble microbiological medium;
introducing a liquid test sample into the sample container to dissolve the microbiological medium; and
forming, in the container, a gelled layer of the dissolved microbiological medium, with no requirement for filtering or concentrating the liquid test sample.

40. The method of claim 39, wherein both the solid nutrient base media component and the solid gelling agent component are coated on the one or more inner surface(s) of the sample container.

41. The method of claim 39 or 40, wherein solid gelling agent component comprises glucomannan derived from Konjac root.

42. The method of any one of claims 39-41, wherein the amount of glucomannan is from about 45% to about 65% (wt./wt.) of the base media and the gelling agent components taken together.

43. The method of claim 42, wherein the amount of glucomannan is from about 50% to about 65% (wt./wt.) of the base media and the gelling agent components taken together.

44. The method of any one of claims 39-43, wherein the amount of glucomannan is from about 4% to about 10% (wt./wt.), from about 5% to about 10% (wt./wt.), or from about 5% to about 7% (wt./wt.) of the liquid test sample.

45. The method of any one of claims 39-44, wherein, in addition to the glucomannan, the gelling agent component comprises at least one of a gum, a natural polymer, and/or synthetic polymer.

46. The method of claim 45, wherein the gum comprises guar gum and/or xanthan gum.

47. The method of any one of claims 39-46, wherein the glucomannan has been heat cured at a temperature and for a time sufficient to remove or substantially remove water therefrom and/or provide adequate sterilization thereof.

48. The method of any one of claims 39-47, wherein the dissolution and forming are at one or more temperature(s) in a range of 10° C. (50° F.) to 45° C. (113° F.).

49. The method of claim 48, wherein the dissolution and forming are at room temperature(s) or ambient temperature(s) in a range of about 16° C. (61° F.) and about 25° C. (77° F.).

50. The method of any one of claims 39-49, as part of a method for enumerating a target organism in the test sample, further comprising:
incubating the gelled layer at one or more incubation temperature(s) and for a time sufficient to provide for microbial growth and detectable microbiological colony formation in or on the gelled medium layer; and
counting the colonies to enumerate a number of target microorganisms in the test sample, wherein less than 1 target organism/mL of test sample may be detected and enumerated.

51. A kit or combination of components for enumerating a target organism, comprising:
a transparent test sample container;
a solid nutrient base media component; and
a solid glucomannan-containing gelling agent component, wherein the solid nutrient base media component and/or the solid gelling agent component is coated on one or more inner surface(s) of the test sample container.

52. The kit or combination of claim 51, wherein both the solid nutrient base media component and the solid gelling agent component are coated on the one or more inner surface(s) of the sample container.

53. The kit or combination of claim 51 or 52, wherein the solid gelling agent component comprises glucomannan derived from Konjac root.

54. The kit or combination of any one of claims 51-53, wherein the amount of glucomannan is from about 45% to about 65% (wt./wt.) of the base media and the gelling agent components taken together.

55. The kit or combination of claim 54, wherein the amount of glucomannan is from about 50% to about 65% (wt./wt.) of the base media and the gelling agent components taken together.

56. The kit or combination of any one of claims 51-55, wherein the amount of glucomannan is from about 4% to about 10% (wt./wt.), from about 5% to about 10% (wt./wt.), or from about 5% to about 7% (wt./wt.) of a liquid test sample testable by the kit.

57. The kit or combination of any one of claims 51-56, wherein, in addition to the glucomannan, the gelling agent component comprises at least one of a gum, a natural polymer, and/or synthetic polymer.

58. The kit or combination of claim 57, wherein the gum comprises guar gum and/or xanthan gum.

59. The kit or combination of any one of claims 51-58, wherein the glucomannan has been heat cured at a temperature and for a time sufficient to remove or substantially remove water therefrom and/or provide adequate sterilization thereof.
